(19) United States
(12) Patent Application Publication
(10) Pub. No.: US 2014/0223670 A1
(43) Pub. Date: Aug. 14, 2014

(54) COLORING METHOD FOR KERATINIC FIBERS

(71) Applicant: Henkel AG & Co. KGaA, Dusseldorf (DE)

(72) Inventors: Aileen Wagner, Hamburg (DE); Stephan Schwartz, Wedel (DE)

(73) Assignee: Henkel AG & Co. KGaA, Dusseldorf (DE)

(21) Appl. No.: 14/243,737

(22) Filed: Apr. 2, 2014

Related U.S. Application Data
(63) Continuation of application No. PCT/EP2013/076076, filed on Dec. 10, 2013.

(30) Foreign Application Priority Data
Dec. 20, 2012 (DE) 102012223936.7

(51) Int. Cl. A61K 8/22; A61K 8/38; A61Q 5/10

(52) U.S. Cl. CPC A61K 8/22; A61Q 5/10; A61K 8/38
USPC 8/406

(57) ABSTRACT

The present specification provides a method for changing the color of keratinic fibers. The method includes producing a ready-to-use agent before application by combining a powdered preparation that includes at least one oxidation dye precursor and at least one pigment that does not change the color of the keratinic fibers with a liquid preparation that includes at least one chemical oxidizing agent. The method also includes mixing the powdered preparation and the liquid preparation. The method also includes distributing the ready-to-use agent on the keratinic fibers, leaving the ready-to-use agent on the keratinic fibers for a period from 1 to 60 minutes, and washing the remaining ready-to-use agent out of the keratinic fibers.

### FIELD OF THE INVENTION

The present invention generally relates to a method for changing the color of keratinic fibers, wherein a ready-to-use agent is produced by mixing two separately packaged preparations before application. The invention also relates to a two-part coloring agent and to a method for predicting the color result of oxidatively-dyed keratinic fibers.

### BACKGROUND OF THE INVENTION

Various methods are used for changing the color of human hair. As a general rule, either substantive dyes or oxidation dyes, which are formed by the oxidative coupling of one or more developer components with one another or with one or more coupler components, may be used to color human hair. These coupler and developer components are known as oxidation dye precursors. The colors obtained with oxidation dyes may be referred to as permanent or semi-permanent colors. Oxidative lightening methods, in which natural or synthetic dyes in the hair fiber are destroyed by oxidation, causing the color to be removed, are mostly used for lightening or bleaching.

These agents mostly contain hydrogen peroxide as the oxidizing agent. As hydrogen peroxide is not sufficiently stable in storage in the alkaline pH range, oxidative coloring and/or lightening agents may include two components, which are mixed together immediately before use. It is important to ensure that the two components are mixed together homogeneously; otherwise uneven coloring results may develop.

Consumers may obtain an indication of the intended hair color for a hair coloring agent via the packaging of the hair coloring agent or a color chart included in the packaging. However, consumers may not consider these indications to be sufficient and/or satisfactory as the result of the coloring may only be seen on completion of the coloring process and subsequent rinsing of the hair fibers. Uncertainties can thus arise when performing a conventional hair coloring, in particular when using an oxidative hair coloring agent for the first time or when intentionally making a major change to the hair color.

The main cause of the uncertainties may result from the color of the ready-to-use hair coloring agent and/or the color of various preparations that have to be mixed together before application to form the actual ready-to-use agent.

The colors of the separately packed preparations in commercial hair coloring kits and the colors of the actual application mixtures may differ greatly from one another and may have no similarity at all to the intended color on the hair. Thus, hair coloring 1) may not be carried out, 2) may not be carried out according to the directions, and/or 3) may have been abandoned too soon, out of fear of achieving an undesired coloring result. As a consequence, hair coloring may need to be repeated, which may be stressful for the hair fibers, as well as being undesirable from a time and cost perspective. Therefore, there is a need for hair coloring methods which allow the user to visualize the achievable hair color throughout the entire application.

Accordingly, the object of the present specification was to optimize oxidative hair coloring methods such that the aforementioned disadvantages can be overcome. In particular, the achievable hair color may be visible and recognizable to the consumer throughout the entire coloring process, so as to prevent the coloring process from being abandoned due to uncertainty.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A method for changing the color of keratinic fibers includes producing a ready-to-use agent before application by combining a powdered preparation that includes at least one oxidation dye precursor and at least one pigment that does not change the color of the keratinic fibers with a liquid preparation that includes at least one chemical oxidizing agent. The method also includes mixing the powdered preparation and the liquid preparation together. The method also includes distributing the ready-to-use agent on the keratinic fibers, leaving the ready-to-use agent on the keratinic fibers for a period from 1 to 60 minutes, and washing any remaining ready-to-use agent out of the keratinic fibers.

An agent for changing the color of keratinic fibers includes a mixture of a powdered coloring preparation that includes at least one oxidation dye precursor and at least one pigment that does not change the color of the keratinic fibers and a liquid preparation comprising at least one chemical oxidizing agent. The powdered coloring preparation and the liquid preparation are separately packaged. The colors of the powdered coloring preparation, the agent, and the colored keratinic fibers have comparable L values, a values, and b values when the colors are measured in the L*a*b* color space.

A method for predicting the color result of oxidatively-dyed keratinic fibers includes providing a powdered preparation that is colored in a shade of a ready-to-use agent and that is colored to a shade that the keratinic fibers will exhibit after coloring. The ready-to-use agent is produced by mixing the powdered preparation that includes at least one oxidation dye precursor and at least one pigment that does not change the color of the keratinic fibers with a liquid preparation that includes at least one chemical oxidizing agent.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Through the selective addition of pigments to a powdered coloring preparation, an approximate indication of both the color of the ready-to-use application mixture and the color that is achievable on the hair fibers may be obtained. In this way it has been possible to provide a hair coloring method in which the color of the powdered coloring preparation is maintained, or remains substantially unchanged, at every stage of the process and thus the achievable hair color is apparent to the user throughout the entire coloring process.

The present specification provides a method for changing the color of keratinic fibers which includes producing a ready-to-use agent immediately before application by combining a powdered preparation that includes at least one oxidation dye precursor and at least one pigment that does not change the color of the keratinic fibers with a liquid preparation that includes at least one chemical oxidizing agent. The method also includes mixing the powdered preparation and the liquid preparation. The method also includes distributing the ready-to-use agent on the keratinic fibers, leaving the ready-to-use agent on the keratinic fibers for a period from 1 to 60 minutes, and washing any remaining ready-to-use agent out of the keratinic fibers.

In order for the achievable hair color to be visible throughout the entire hair coloring process, it may be advantageous if there is little or no visual difference between the color of the powdered preparation and both 1) the color of the ready-to-use agent and 2) the achievable color of the keratinic fibers.

Accordingly, during the coloring method, the color of the powdered preparation should at most change in terms of its lightness and/or intensity. The actual shade of the powdered preparation, however, should ideally be comparable with the shade of both 1) the ready-to-use agent and 2) the colored keratinic fibers.

As used in the present specification and in the appended claims, the term “comparable” indicates little or no difference between the colors of the powdered preparation, the ready-to-use agent, and the colored keratin fibers. More specifically, the individual L values, a values and b values of the colors of the powdered preparation, the ready-to-use agent, and the colored keratin fibers differ only slightly when the colors are measured in the L*a*b* color space. For example, in a preferred example, after any remaining ready-to-use agent has been washed from the keratinic fibers, the colors of the powdered preparation, the ready-to-use agent and the keratinic fibers each have comparable L values, a values and b values when the colors are measured in the L*a*b* color space.

In a particularly preferred example, after any remaining ready-to-use agent has been washed from the keratinic fibers, the L value, a value and b value of the color of the keratinic fibers differ from the L values, a values and b values of the color of the powdered preparation, the color of the ready-to-use agent or combinations thereof by a maximum of 30%, preferably by a maximum of 25%, more preferably by a maximum of 20% and in particular by a maximum of 15%, the L, a and b values relating to the coordinate system of the L*a*b* color space.

The L*a*b* color space refers to the CIELAB color space. The L value may represent the lightness of the color (black-white axis); the higher the L value, the lighter the color. The a value may represent the red-green axis of the system; the higher the value, the greater the shift to the red axis. The b value may represent the yellow-blue axis of the system; the higher the value, the greater the shift to the yellow axis.

As used in the present specification and in the appended claims, the term “keratinic fibers,” “keratin-containing fibers,” or similar terms may refer to wool, fur, feathers, and in particular human hair. The coloring and/or lightening methods according to the present specification may, in principle, also be used for application on other natural fibers, such as for example cotton, jute, sisal, linen, silk or modified natural fibers, such as for example regenerated cellulose, nitrocellulose, alkyl or hydroxyalkyl cellulose or acetyl cellulose.

The method may include in the first process step mixing a powdered (color-changing) preparation and a liquid (oxidizing agent) preparation. Mixing of the two preparations preferably may take place in a re-sealable container.

The color-changing preparation may be in a powder form. As used in the present specification and in the appended claims a “powder form” refers to a solid, free-flowing presentation form comprising individual particles. The individual particles may have differing particle sizes from 1 μm to a maximum of 1 mm. Powders in which the individual particles have as homogeneous a particle size as possible may be preferred. The particles may also be adapted in terms of their particle size to the requirements of the agents by means of physical treatment, such as screening, compressing, granulating or pelletizing, or by the addition of certain auxiliary substances, to allow for example a better solution or dispersion of the color-changing preparation.

The powdered preparation may contain at least one oxidation dye precursor, which refers to preferably one or more developer components and optionally one or more coupler components.

It may be preferable according to the present specification to select as the developer component at least one compound from the group which is formed from p-phenylenediamine, p-toluoylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraminopyrimidine, 4-hydroxy-2,5,6-triaminopyrimidine, 2-hydroxy-4,5,6-triaminopyrimidine, and the physiologically acceptable salts thereof.

The developer components are preferably used in an amount from 0.001 to 20 wt. %, preferably from 0.005 to 10 wt. %, and in particular from 0.01 to 5 wt. %, relative in each case to the ready-to-use agent.

In the context of oxidative dyeing, coupler components do not develop significant color on their own but always need the presence of developer components. It is therefore preferable according to the present specification, that if at least one developer component is used, then at least one coupler component is additionally used.

Coupler components that are preferred according to the present specification are selected from 3-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2-hydroxyethyl)amino-2-methylphenol, 3-(diethylamino)phenol, N-cyclopentyl-3-aminophenol, 1,3-dihydroxy-5-(methylamino)benzene, 3-ethylamino-4-methylphenol, 2,4-dichloro-3-aminophenol, 2-(2,4-diaminophenoxy)ethanol, 1,3-bis-(2,4-diaminophenoxy)propane, 1-methoxy-2-amino-4-(2-hydroxyethylamino)benzene, 1,3-bis-(2,4-diaminophenyl)propane, 2,6-bis-(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, resorcinol monomethyl ether, 2-methylresorcinol, 5-methylresorcinol, 2,5-dimethylresorcinol, 2-chlororesorcinol, 4-chlororesorcinol, pyrogallol, 1,2,4-trihydroxybenzene, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 2,6-dihydroxy-3,4-dimethylpyridine, 3,4-diaminopyridine, 2-(2-methoxyethyl)amino-3-amino-6-methoxypyridine, 2-(4′-methoxyphenyl)amino-3-aminopyridine, 1-naphthene, 2-methyl-1-naphthene, 2-hydroxymethyl-1-naphthene, 2-hydroxyethyl-1-naphthene, 1,3-dihydroxynaphthalene, 1,5-dihydroxynaphthalene, 1,6-dihydroxynaphthalene, 1,7-dihydroxynaphthalene, 1,8-dihydroxynaphthalene, 2,7-dihydroxynaphthalene, 2,3-dihydroxynaphthalene, 4-hydroxyindole, 6-hydroxyindole, 7-hydroxyindole, 4-hydroxyindoline, 6-hydroxyindoline, 7-hydroxyindoline, 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine or mixtures of these compounds or physiologically acceptable salts thereof.

The coupler components are preferably used in an amount from 0.001 to 20 wt. %, preferably from 0.005 to 10 wt. %, and in particular from 0.01 to 5 wt. %, relative in each case to the ready-to-use agent.

Developer components and coupler components may be used in approximately equimolar amounts to one another. Even if the equimolar amounts have proven convenient a certain excess of individual oxidation dye precursors is not disadvantageous, such that developer components and coupler components may be in a molar ratio of 1:0.5 to 1:3, in particular 1:1 to 1:2.

The powdered preparation may contain at least one pigment that does not change the color of the keratinic fibers. This indicates that the coloring of the hair fibers during the hair coloring process of the present specification should not come about through the pigments in the powdered coloring preparation. The coloring of the keratinic fibers by the oxidation dye precursors contained in the powdered preparation should be neither influenced nor impaired by the pigments.

Pigments that are suitable according to the present specification may be selected, for example, from all pigments that do not color keratinic fibers and that are suitable for cosmetic use.

Colored, special-effect and/or pearlescent pigments are preferred, which may be of organic and/or inorganic origin.

The use of inorganic pigments is particularly preferred because of their excellent light resistance, weather resistance and/or temperature resistance.

The preferred average particle size of the pigments, preferably inorganic pigmentsis from 0.1 μm to 1 mm, more preferably from 0.5 μm to 750 μm and in particular from 10 μm to 500 μm.

Pigments or colored pigments that are suitable according to the present specification give the powdered preparation coloring effects, and are optionally combined with a gloss effect (colored gloss pigments).

Preferred pigments are selected from inorganic pigments, which may be of a natural origin. Inorganic pigments of natural origin may be produced for example from chalk, ochre, umber, green earth, burnt sienna or graphite.

Furthermore, white pigments such as titanium dioxide or zinc oxide, black pigments such as iron oxide black, colored pigments such as ultramarine or iron oxide red, and fluorescent or phosphorescent pigments may be used as inorganic pigments; at least one pigment preferably being a colored, non-white pigment. Metal oxides, metal hydroxides and metal oxide hydrates, mixed-phase pigments, sulfur-containing silicates, metal sulfides, complex metal cyanides, metal sulfates, metal chromates and/or metal molybdates are particularly suitable. Pigments that are preferred in particular are titanium dioxide (CI 77891), black iron oxide (CI 77499), yellow iron oxide (CI 77492), red and brown iron oxide (CI 77491), manganese violet (CI 77742), ultramarine (sodium aluminum sulfosilicate, CI 77007, Pigment Blue 29), chromium oxide hydrate (CI 77289), iron blue (ferric ferrocyanide, CI 77510) and/or carmine (cochineal).

As used in the present specification and in the appended claims suitable special-effect pigments refer to preferably metal special-effect pigments such as bronze pigments.

Further pigments that are preferred according to the present specification include pearlescent pigments. These are usually based on mica and may be coated with one or more of the aforementioned metal oxides or with a metal oxychloride such as bismuth oxychloride. Pearlescent pigments based on natural mica and on natural mica/metal oxide are preferred according to the present specification.

Micas are phyllosilicates. The most important representatives of these silicates are muscovite, phlogopite, paragonite, biotite, lepidolite and margarite. In order to produce pearlescent pigments in combination with metal oxides the mica, predominantly muscovite or phlogopite, is coated with a metal oxide.

As an alternative to natural mica, synthetic mica coated with one or more metal oxide(s) may optionally also be used as a pearlescent pigment. Such suitable pearlescent pigments based on natural micas are described in the patent application WO 2005/065632, to which reference is expressly made. Particularly preferred pearlescent pigments are based on natural or synthetic mica and are coated with one or more of the aforementioned metal oxides.

The color of the individual pigments may be varied by altering the coating thickness of the metal oxide(s).

Examples of particularly suitable pearlescent pigments are commercially available under the trade names Rona®, Colorona®, Dichrona® and Timiron® from Merck, Ariabel® from Sensient, Prestige® from Eckart Cosmetic Colors and Sunshine® from Sunstar.

In a preferred example the powdered preparation contains as the pigment, at least one inorganic colored pigment, special-effect pigment, and/or pearlescent pigment which is preferably selected from metal oxides, metal hydroxides, metal oxide hydrates, sulfur-containing silicates, metal sulfides, complex metal cyanides, metal sulfates, bronze pigments and/or from mica-based pigments which are coated with at least one metal oxide and/or a metal oxychloride.

In a particularly preferred example, the powdered preparation contains as the pigment at least one inorganic colored and/or pearlescent pigment selected from titanium dioxide (CI 77891), black iron oxide (CI 77499), yellow iron oxide (CI 77492), red and/or brown iron oxide (CI 77491), manganese violet (CI 77742), ultramarine (sodium aluminum sulfosilicate, CI 77007, Pigment Blue 29), chromium oxide hydrate (CI 77289), iron blue (ferric ferrocyanide, CI 77510) and/or from mica pigments which are coated with one or more of the cited metal oxides.

Powdered preparations containing one or more mica pigments coated with titanium dioxide (CI 77891), black iron oxide (CI 77499), red and/or brown iron oxide (CI 77491) and/or ultramarine (sodium aluminum sulfosilicate, CI 77007, Pigment Blue 29) are preferred in particular.

In a further particularly preferred example, the powdered preparation contains, relative to its weight, 0.05 to 45 wt. %, preferably 0.1 to 40 wt. %, particularly preferably 0.2 to 35 wt. % and in particular 0.3 to 30 wt. % of at least one oxidation dye precursor, and 1 to 40 wt. %, preferably 3 to 35 wt. %, particularly preferably 5 to 30 wt. % and in particular 7.5 to 25 wt. % of at least one pigment that does not change the color of the fibers.

In a further preferred example of the method according to the present specification, the color-changing preparation includes at least one solid inorganic alkalizing agent. As used in the present specification and in the appended claims the term “solid inorganic alkalizing agent” refers to salts that have an alkaline reaction in an aqueous environment. Suitable solid inorganic alkalizing agents according to the present specification are preferably selected from the group formed from sodium hydroxide, potassium hydroxide, calcium hydroxide, magnesium hydroxide, magnesium carbonate hydroxide, barium hydroxide, sodium phosphate, potassium phosphate, sodium silicate, potassium silicate, ammonium carbonate, sodium carbonate, potassium carbonate and magnesium carbonate.

The inorganic alkalizing agent is preferably present in the powdered color-changing preparation in an amount that is sufficient to give the ready-to-use agent an alkaline pH, preferably a pH from 8 to 12.

In a preferred example, the inorganic alkalizing agent is present in the powdered color-changing preparation in an amount that is sufficient to give the powdered preparation a pH from 8 to 10 and in particular a pH from 8.5 to 9.5.

To reduce dust formation and to improve the flowability of the powdered color-changing preparation, it may be advantageous to add an oily component to the powdered preparation. Paraffin oil has proved to be particularly preferred here. Therefore, a further example may be characterized in that the color-changing preparation additionally contains at least one paraffin oil.

Paraffin oil is preferably included in a proportion from 0.01 to 5 wt. %, in particular from 0.05 to 3 wt. %, and particularly from 0.08 to 1.0 wt. %, relative in each case to the ready-to-use agent.

In the method according to the present specification, the oxidizing agent preparation may be a liquid. As used in the present specification and in the appended claims “liquid” refers to a state of aggregation which is liquid at room temperature and which has a viscosity that makes it possible for the preparation to be poured.

The oxidizing agent preparation according to the present specification preferably contains the chemical oxidizing agent(s) in a cosmetically acceptable carrier. This cosmetic carrier is preferably aqueous, alcoholic or aqueous-alcoholic. Within the meaning of the present specification an aqueous carrier preferably contains at least 40 wt. %, in particular at least 50 wt. %, of water. As used in the present specification and in the appended claims “aqueous-alcoholic carriers” refers to hydrous compositions preferably containing 3 to 70 wt. % of a C₁ to C₄ alcohol, in particular ethanol or isopropanol. The liquid preparation may additionally contain further organic solvents, such as for example 4-methoxybutanol, ethyl diglycol, 1,2-propylene glycol, n-propanol, n-butanol, n-butylene glycol, glycerol, diethylene glycol monoethyl ether, and diethylene glycol mono-n-butyl ether.

The oxidizing agent preparation may contain at least one chemical oxidizing agent. This is understood to be a chemical oxidizing agent which is different from atmospheric oxygen.

Preferred chemical oxidizing agents include hydrogen peroxide and/or a solid addition product of hydrogen peroxide with inorganic and/or organic compounds.

Persulfates, chlorites and in particular hydrogen peroxide or addition products thereof with urea, melamine and sodium borate are particularly preferred. In particular, the oxidizing agent preparation preferably contains hydrogen peroxide as the chemical oxidizing agent.

The ready-to-use agent preferably contains the chemical oxidizing agent, in particular hydrogen peroxide, in a proportion from 0.5 to 12 wt. %, in particular 1 to 10 wt. %, and particularly preferably from 2 to 8 wt. %, relative in each case to the total weight of the ready-to-use agent.

It has proved advantageous for the oxidizing agent preparation to contain at least one non-ionic surfactant. Ethoxylated non-ionic surfactants are preferably used here. Therefore, a further example of the present specification is characterized in that the oxidizing agent preparation additionally contains at least one non-ionic surfactant.

Alkylene oxide addition products with saturated linear fatty alcohols, fatty acid esters and fatty acids each containing 2 to 80 mol of ethylene oxide and/or 1 to 5 mol of propylene oxide per mol of fatty alcohol or fatty acid are preferred non-ionic surfactants. Preparations having outstanding properties are likewise obtained if they contain fatty acid esters of ethoxylated glycerol as non-ionic surfactants.

It has proved particularly advantageous for the further non-ionic surfactant to have an HLB value above 10, preferably above 14. To this end, the non-ionic surfactant has a sufficiently high degree of ethoxylation. Therefore, a further example of the present specification is characterized in that the liquid preparation contains, as the non-ionic surfactant, at least one ethoxylated surfactant having at least 30 ethylene oxide units.

In particular, in addition to the correspondingly ethoxylated fatty alcohols, the addition products of 30 to 60 mol of ethylene oxide with castor oil and hydrogenated castor oil are particularly suitable according to the present specification.

Examples of such suitable surfactants have the INCI names Steareth-30, Ceteareth-30, Oleth-30, Ceteareth-50 or PEG-40 Hydrogenated Castor Oil and PEG-60 Hydrogenated Castor Oil.

In a preferred example, the aforementioned ethoxylated non-ionic surfactant(s) may also be used in the oxidizing agent preparation as a mixture with fatty alcohols and/or ionic surfactants.

Preferred examples of such mixtures include mixtures of fatty alcohols, ethoxylated non-ionic surfactants, in particular ethoxylated hydrogenated castor oils and anionic sulfate surfactants. Such a commercial product is available for example from BASF under the name Emulgade® F (INCI name: Cetearyl Alcohol, PEG-40 Castor Oil, Sodium Cetearyl Sulfate).

The additional non-ionic surfactant(s) may preferably be included in the ready-to-use agents in amounts from 0.1 to 10 wt. %, preferably from 0.5 to 8 wt. %, and particularly preferably from 1.0 to 5.0 wt. %, relative in each case to the ready-to-use agent.

To stabilize the oxidizing agent in the liquid oxidizing agent preparation it is preferable to use complexing agents. Complexing agents are substances that are capable of complexing metal ions. Preferred complexing agents are chelating agents, in other words substances which form cyclic compounds with metal ions, wherein an individual ligand occupies more than one coordination site on a central atom. The number of bound ligands is dependent on the coordination number of the central ion. Common chelating agents which are preferred in the context of the present specification are for example polyoxycarboxylic acids, polyamines, ethylenediaminetetraacetic acid (EDTA), nitrilotriacetic acid (NTA) and hydroxyethane diphosphonic acids or the alkali salts thereof. Complexing agents that are preferred according to the present specification are phosphonates, preferably hydroxyalkane or aminoalkane phosphonates, and in particular 1-hydroxyethane-1,1-diphosphonate (HEDP) or the disodium or tetrasodium salt thereof and/or ethylenediamine tetramethylene phosphonate (EDTMP) or the hexasodium salt thereof and/or diethylenetriamine pentamethylene phosphonate (DTPMP) or the heptasodium or octasodium salt thereof. Dipicolinic acid is also preferably used according to the present specification as the complexing agent. Agents containing a combination of an EDTA salt and HEDP and dipicolinic acid are particularly preferred according to the present specification.

As a further constituent, the preparations according to the present specification can preferably contain at least one ammonium compound from the group comprising ammonium chloride, ammonium carbonate, ammonium bicarbonate, ammonium sulfate and/or ammonium carbamate, preferably in an amount from 0.5 to 50 wt. %, more preferably from 1 to 40 wt. %, and in particular from 5 to 30 wt. %, relative to the total composition of the agent.

The ready-to-use agents may moreover contain further active ingredients, auxiliary substances and additives, for example non-ionic polymers, such as vinyl pyrrolidinone/vinyl acrylate copolymers, polyvinyl pyrrolidinone, vinyl pyrrolidinone/vinyl acetate copolymers, polyethylene glycols and polysiloxanes; additional silicones such as volatile or non-volatile, straight-chain, branched or cyclic, cross-linked or uncrosslinked polyalkyl siloxanes (such as dimethicones or cyclomethicones), polyaryl siloxanes and/or polyalkylaryl siloxanes, in particular polysiloxanes having organofunctional groups, such as substituted or unsubstituted amines (amodimethicones), carboxyl, alkoxy and/or hydroxyl groups (dimethicone copolyols), linear polysiloxane(A)-polyoxyalkylene(B) block copolymers, grafted silicone polymers; anionic polymers, such as acrylic acid homo- and copolymers, xanthan, sodium carboxymethylcellulose; zwitterionic and amphoteric polymers; structuring agents such as glucose, maleic acid and lactic acid, hair-conditioning compounds such as phospholipids, for example lecithin and cephalins; perfume oils, dimethyl isosorbide and cyclodextrins; active ingredients to improve the fiber structure, in particular mono-, di- and oligosaccharides such as for example glucose, galactose, fructose, fruit sugars and lactose; dyes for coloring the agent; amino acids and oligopeptides, in particular arginine and/or serine; protein hydrolysates of animal and/or plant origin, such as for example elastin, collagen, keratin, silk and milk protein hydrolysates, or almond, rice, pea, potato and wheat protein hydrolysates, as well as those in the form of their fatty acid condensation products or optionally anionically or cationically modified derivatives thereof; vegetable oils, such as macadamia nut oil, palm oil, amaranth seed oil, peach kernel oil, avocado oil, olive oil, coconut oil, rapeseed oil, sesame oil, jojoba oil, soybean oil, groundnut oil, evening primrose oil and tea tree oil; light stabilizers; active ingredients such as panthenol, pantothenic acid, pantolactone, allantoin, pyrrolidinone carboxylic acids and salts thereof as well as bisabolol; polyphenols, in particular hydroxycinnamic acids, 6,7-dihydroxycoumarins, hydroxybenzoic acids, catechins, tannins, leukoanthocyanidins, anthocyanidins, flavanones, flavones and flavonols; ceramides or pseudoceramides; vitamins, pro-vitamins and vitamin precursors, in particular from groups A, B₃, B₅, B₆, C, E, F and H; plant extracts; fats and waxes such as fatty alcohols, beeswax, montan wax and paraffins; swelling and penetrating substances such as glycerol, propylene glycol monoethyl ether, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate and PEG-3 distearate as well as pigments.

These further substances may be selected in accordance with the desired properties of the agents. With regard to further optional components and to the amounts of these components used, reference is expressly made the manual “Grundlagen and Rezepturen der Kosmetica, 2^{nd} Edition, Kh. Schrader, Hüthig Buch Verlag, Heidelberg, 1989.

The ready-to-use agent preferably has an alkaline pH, preferably an alkaline pH between 7 and 12, more preferably between 8 and 11.5, and in particular between 8.5 and 11.0. The pH may be adjusted using pH adjusters. Acidifying and alkalizing agents used in cosmetics may be used to adjust the pH. To stabilize the oxidizing agent during storage it is preferable in particular if the oxidizing agent preparation has an acid pH before mixing, in particular between 2.5 and 5.5, preferably between 3.0 and 5.0. Preferred acidifying agents are food acids, such as for example citric acid, acetic acid, malic acid or tartaric acid, as well as dilute mineral acids.

The ready-to-use agent is preferably produced by combining a powdered color-changing preparation and a liquid oxidizing agent preparation in a re-sealable container and then mixing them.

It has proved advantageous for a homogeneous mixing of the two preparations if the proportion by weight of the oxidizing agent in the total weight of the ready-to-use agent is greater than the proportion by weight of the color-changing preparation. A weight ratio of at least 5 is particularly preferred, i.e. one part by weight of the color-changing preparation mixed with at least 5 parts by weight of the liquid oxidizing agent preparation. Therefore, a preferred example of the method according to the present specification is characterized in that the weight ratio between the liquid oxidizing agent preparation and the color-changing preparation has a value greater than 5, preferably greater than 6, and particularly preferably greater than 7.5.

For a good distributability of the ready-to-use agent on the keratinic fibers during distribution on the keratinic fibers, it has proved advantageous for the ready-to-use agent to have an adequate viscosity. As used in the present specification and in the appended claims “adequate viscosity” indicates 1) that the agent is sufficiently liquid to be able to be evenly distributed on the keratinic fibers and 2) that the agent is sufficiently viscous for it to remain on the keratinic fibers rather than dripping off them.

Combined mixtures (comprising the powdered-preparation and the liquid preparation) having a viscosity from 50 to 5000 mPas (measured at 22° C. in a Brookfield RV-T viscometer with an LV-1 or RV-1 spindle and a speed of 30 rpm) have proved particularly preferable. A viscosity from 100 to 4000 mPas, measured under the stated conditions, is particularly preferred according to the present specification. A viscosity from 200 to 3000 mPas, in particular from 300 to 2500 mPas (measured in each case under the aforementioned conditions) is most particularly preferred according to the present specification.

According to a method described in the present specification, the ready-to-use agent is distributed on the keratinic fibers. In one method for changing the color of human hair, the ready-to-use agent may be distributed directly on the user's hair. The distribution preferably takes place manually. To this end, the user may remove the ready-to-use agent from the mixing container, preferably from the re-sealable container, by scooping or pouring it onto the hand and then distributing the agent on the hair and preferably working it in to the hair. Direct contact between the ready-to-use agent and the hands is preferably avoided here by using suitable gloves such as disposable gloves, made from latex for example.

According to the method described in the present specification, the ready-to-use agent remains on the keratinic fibers to be treated for a period from 1 to 60 minutes. The period is preferably between 10 and 45 minutes.

The application temperatures may be in a range between 15 and 40° C. During the time for which the ready-to-use agent remains on the keratinic fibers, a higher or precisely defined temperature may optionally also be set using external heat sources. It is particularly preferable for the color change to be supported by physical measures. Methods according to the present specification in which the application is supported by the action of heat, infrared and/or UV radiation during the contact time may be preferred.

At the end of the contact time, the ready-to-use agent or any remaining ready-to-use agent may be removed from the keratinic fibers to be treated by rinsing. To this end the keratinic fibers may be rinsed with water and/or an aqueous surfactant preparation. Water heated to a temperature of 20° C. to 40° C. or a correspondingly heated aqueous surfactant preparation may be used for this purpose.

The present specification also describes an agent for changing the color of keratinic fibers, which can be produced from two separately packaged preparations by mixing the two preparations. One of the two preparations may be a powdered coloring preparation containing at least one oxidation dye precursor and at least one pigment that does not change the color of the fibers. The other of the two preparations may be a liquid preparation, containing at least one chemical oxidizing agent. In one example, the colors of the powdered coloring preparation, the ready-to-use agent for changing the color of keratinic fibers, and the colored keratinic fibers each have comparable L values, a values and b values when the colors are measured in the L*a*b* color space.

All that has been stated in respect of the method according to the present specification applies with necessary alterations to further preferred examples of the agents according to the present specification.

The present invention thirdly provides a method for predicting the color result of oxidatively-dyed keratinic fibers, in which a ready-to-use agent is produced by mixing a powdered preparation containing at least one oxidation dye precursor and at least one pigment that does not change the color of the fibers with a liquid preparation containing at least one chemical oxidizing agent. The method may be characterized in that the powdered preparation is already colored in the shade that the ready-to-use agent and the keratinic fibers will also exhibit after coloring.

All that has been stated in respect of the method according to the present specification applies with necessary alterations to further preferred examples of the method according to the present specification.

### EXAMPLES

The following preparations were produced. Table 1 below contains examples of a number of powdered preparations (I-VI) according to the present specification. Table 2 contains an example of a liquid oxidizing agent preparation (E1) according to the present specification. Unless otherwise specified, the stated amounts are percentages by weight.

TABLE 1

| Raw materials | I | II | III | IV | V | VI |
|---|---|---|---|---|---|---|
| Sodium metasilicate | 22.73 | 22.73 | 22.73 | 22.73 | 22.73 | 22.73 |
| FE, anhydrous | | | | | | |
| Luvomag ® C 013 | 9.15 | 9.15 | 13.77 | 9.10 | 9.10 | 11.40 |
| Perfume | 0.60 | 0.60 | 0.60 | 0.60 | 0.60 | 0.60 |
| Liquid paraffin | 1.65 | 1.65 | 1.65 | 1.65 | 1.65 | 1.65 |
| p-Toluylenediamine | 17.60 | 14.3 | 1.50 | 4.79 | 3.56 | 1.97 |
| sulfate | | | | | | |
| Resorcinol | 5.73 | 4.91 | 1.25 | 1.57 | 0.99 | 0.22 |
| 3-Aminophenol | 2.40 | 1.85 | 0.65 | 0.37 | 0.30 | 0.06 |
| 2,4-Diaminophenoxy- | 1.53 | 0.76 | 2.40 | | | |
| ethanol 2HCI | | | | | | |
| 4,5-Diamino-1-(2- | | | 5.10 | | | |
| hydroxyethyl)pyrazole | | | | | | |
| sulfate | | | | | | |
| 2-Methylresorcinol | | | | 0.20 | 0.17 | 0.18 |
| 2-Amino-3- | | | | 0.34 | | |
| hydroxypyridine | | | | | | |
| 2,7-Dihydroxynaph- | | | | | | 0.27 |
| thalene | | | | | | |
| 4-Chlororesorcinol | | | | | 0.43 | 0.48 |
| Arialbel ® Blue | | | 4.50 | | | |
| Prestige ® Fire Red | | | 4.50 | | | |
| Colorona ® Blackstar | | 13.60 | | 11.40 | 11.40 | |
| Gold | | | | | | |
| Colorona ® Mica | 13.60 | | | | | |
| Black | | | | | | |
| Colorona ® Precious | | | | | | 9.10 |
| Gold | | | | | | |
| Timiron ® Diamond | 4.50 | 4.50 | 4.50 | 4.50 | 4.50 | 4.50 |
| MP 149 | | | | | | |
| Ammonium chloride | to 100 | | | | | |
| Color of the powdered | black | dark | red- | brown | brown | gold |
| preparations | | brown | violet | | | |
| pH of the powdered | 8.8- | 8.8- | 8.8- | 8.8- | 8.8- | 8.8- |
| preparations | 9.3 | 9.3 | 9.3 | 9.3 | 9.3 | 9.3 |

TABLE 2

| Raw material | E1 |
|---|---|
| EDTA Na₂ | 0.15 |
| Disodium pyrophosphate | 0.30 |
| Sodium benzoate | 0.04 |
| Emulgade ® F | 2.10 |
| Hydrogen peroxide (aqueous, 50%) | 6.00 (or 12.00) |
| Phosphoric acid | 0.04 |
| Water | to 100 |

Below is given more detail concerning the raw materials indicated in Tables 1 and 2. Luvomag C 013 (INCI name: Magnesium Carbonate Hydroxide), Lehmann & Voss; Arialbel® Blue (INCI name: CI 77007 (Ultramarine S), Silicic acid aluminum sodium salt, sulfurized, Pigment Blue 29), Sensient; Prestige® Fire Red (INCI name: Mica, CI 77491 (Iron Oxides)), Eckart Cosmetic Colors; Colorona® Blackstar Gold (INCI name: Mica, CI 77499 (Iron Oxides)), Merck; Colorona® Mica Black (INCI name: CI 77499 (Iron Oxides) Mica, CI 77891 (Titanium oxides)), Merck; Colorona® Precious Gold (INCI name: Mica, CI 77891 (Titanium oxides), Silica, CI 77499 (Iron Oxides), SnO), Merck; Timiron® Diamond MP 149 (INCI name: Mica, CI 77891 (Titanium oxides)), Merck; Emulgade® F (INCI name: Cetearyl Alcohol, PEG-40 Castor Oil, Sodium Cetearyl Sulfate), BASF.

In the example, 11 g of powdered color-changing preparation 1 and 11 g of powdered color-changing preparation VI were each mixed with 100 g of the oxidizing agent preparation (E1) (3% hydrogen peroxide) in a re-sealable mixing container by means of vigorous, uninterrupted shaking (40 times) to form two ready-to-use agents.

The ready-to-use agent based on preparation I was colored black, while the ready-to-use agent based on preparation VI was colored gold. The ready-to-use agents were each removed from the containers using a cupped hand and distributed evenly over two identical strands of hair (Alkino).

Each ready-to-use agent remained on the hair strands for a contact time of 30 minutes at room temperature.

In both cases, any remaining agent was rinsed out of the hair strands for approximately 2 minutes using tepid water and the strands were dried with a towel.

Even, long-lasting and glossy colors having high color intensity and chromaticity were obtained. The hair strand colored from preparation I was black in color. The hair strand colored from preparation VI was gold-blond in color.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for changing the color of keratinic fibers, the method comprising:
producing a ready-to-use agent before application by combining:
a powdered preparation comprising:
at least one oxidation dye precursor; and
at least one pigment that does not change the color of the keratinic fibers; and
a liquid preparation, comprising at least one chemical oxidizing agent; and
mixing the powdered preparation and the liquid preparation;
distributing the ready-to-use agent on the keratinic fibers;
leaving the ready-to-use agent on the keratinic fibers for a period from 1 to 60 minutes; and
washing any remaining ready-to-use agent out of the keratinic fibers.

2. The method of claim 1, in which after any remaining ready-to-use agent has been washed out, the colors of the powdered preparation, the ready-to-use agent, and the keratinic fibers each have comparable L values, a values, and b values as measured in the L*a*b* color space.

3. The method of claim 1, in which after any remaining ready-to-use agent has been washed out, an L value, an a value, and a b value of the color of the keratinic fibers differs from an L value, an a value and a b value of the color of the powdered preparation, the ready-to-use agent, or a combination thereof, by a maximum of 30% as measured in the L*a*b* color space.

4. The method of claim 1, in which the at least one pigment comprises an inorganic colored pigment, a special-effect pigment, a pearlescent pigment, or combinations thereof selected from the group consisting of metal oxides, metal hydroxides, metal oxide hydrates, sulfur-containing silicates, metal sulfides, complex metal cyanides, metal sulfates, bronze pigments, and mica-based pigments which are coated with at least one metal oxide, at least one metal oxychloride or combinations thereof.

5. The method of claim 4, in which the inorganic colored pigment, pearlescent pigment, or combinations thereof are selected from the group consisting of titanium dioxide (CI 77891), black iron oxide (CI 77499), yellow iron oxide (CI 77492), red iron oxide, brown iron oxide (CI 77491), manganese violet (CI 77742), ultramarine (sodium aluminum sulfosilicate, CI 77007, Pigment Blue 29), chromium oxide hydrate (CI 77289), iron blue (ferric ferrocyanide, CI 77510), and mica pigments which are coated with one or more metal oxides.

6. The method of claim 1, in which the powdered preparation comprises, relative to its weight:
0.05 to 45 wt. % of the at least one oxidation dye precursor; and
1 to 40 wt. % of the at least one pigment.

7. The method of claim 1, in which the powdered preparation further comprises a solid inorganic alkalizing agent selected from the group consisting of sodium hydroxide, potassium hydroxide, calcium hydroxide, magnesium hydroxide, magnesium carbonate hydroxide, barium hydroxide, sodium phosphate, potassium phosphate, sodium silicate, potassium silicate, ammonium carbonate, sodium carbonate, potassium carbonate, and magnesium carbonate.

8. The method of claim 1, in which the chemical oxidizing agent comprises, hydrogen peroxide, a solid addition product of hydrogen peroxide with organic compounds, a solid addition product of hydrogen peroxide with inorganic compounds, or combinations thereof.

9. An agent for changing the color of keratinic fibers, the agent comprising:
a mixture of:
a powdered coloring preparation comprising:
at least one oxidation dye precursor; and
at least one pigment that does not change the color of the keratinic fibers; and
a liquid preparation comprising at least one chemical oxidizing agent;
in which the powdered coloring preparation and the liquid preparation are separately packaged and in which the colors of the powdered coloring preparation, the agent, and colored keratinic fibers have comparable L values, a values, and b values as measured in the L*a*b* color space.

10. The agent of claim 9, in which after use, an L value, a value and b value of the color of the keratinic fibers differs from an L value, a value and b value of the color of the powdered coloring preparation, the ready-to-use agent, or a combination thereof by less than 30% as measured in the L*a*b* color space.

11. The agent of claim 9, in which:
the at least one pigment comprises an inorganic colored pigment, a special-effect pigment, a pearlescent pigment, or combinations thereof;
the powdered coloring preparation comprises:
0.05 to 45 wt. % of the at least one oxidation dye precursor; and
1 to 40 wt. %, of the at least one pigment; and
the powdered preparation comprises a solid inorganic alkalizing agent.

12. The agent of claim 9, in which the at least one pigment has an average particle size from 0.1 μm to 1 mm.

13. A method for predicting the color result of oxidatively-dyed keratinic fibers, the method comprising:
providing a powdered preparation that is colored in the shade of a ready-to-use agent and that is colored to the shade that the keratinic fibers will exhibit after coloring;
producing a ready-to-use agent by mixing the powdered preparation comprising at least one oxidation dye precursor and at least one pigment that does not change the color of the keratinic fibers with a liquid preparation comprising at least one chemical oxidizing agent.

14. The method of claim 13, in which:
the at least one pigment comprises an inorganic colored pigment, a special-effect pigment, a pearlescent pigment, or combinations thereof;
the powdered coloring preparation comprises:
0.05 to 45 wt. % of the at least one oxidation dye precursor; and
1 to 40 wt. %, of the at least one pigment; and
the powdered preparation comprises a solid inorganic alkalizing agent.

15. The method of claim 13, in which after use, an L value, a value, and b value of the color of the keratinic fibers differs from an L value, a value and b value of the color of the powdered preparation, the ready-to-use agent, or a combination thereof, by a maximum of 30% as measured in the L*a*b* color space.
